(19) United States
(12) Patent Application Publication
(10) Pub. No.: US 2016/0295944 A1
(43) Pub. Date: Oct. 13, 2016

(54) HAIRWEAR EQUIPPED WITH MULTI-LAYER STRUCTURES

(71) Applicant: SECRETWOMAN CO., LTD., Seo-gu Daejeon (KR)

(72) Inventor: Yeong-Hyu KIM, Daejeon (KR)

(21) Appl. No.: 14/652,116

(22) PCT Filed: May 12, 2014
(86) PCT No.: PCT/KR2014/004209

§ 371 Date: Jun. 14, 2015

(30) Foreign Application Priority Data
Nov. 1, 2013 (KR) 10-2013-0132397

(51) Int. Cl. A41G 3/00; A61B 5/00; A41G 5/00

(52) U.S. Cl. CPC A41G 3/0041; A41G 5/0073; A61B 5/6803

(57) ABSTRACT

A hairwear has multi-layer structures. One or more multi-layered hair lines protruding onto the top of a reference plane made by a front portion and base hair lines or a cap-shaped transplant portion are formed. The hairwear includes a front portion including a skin color portion, and a clip fixing portion formed at both sides of the skin color portion; and one or more base hair lines both ends of which are fixed and combined to the front portion. The multi-layered hair lines both ends of which are fixed and combined to the front portion and which protrude onto the top of the reference plane made by the front portion and the base hair lines are formed and the multi-layered hair lines are made of a material capable of fixing the shape and the position.

### BACKGROUND OF THE INVENTION

1. Field of the Invention

The present invention relates to a hairwear equipped with multi-layer structures, and more particularly to a hairwear equipped with hair lines having multi-layer structures characterized in that one or more multi-layered hair lines protruding onto the top of a reference plane made by a front portion and base hair lines or a cap-shaped transplant portion are formed.

2. Description of the Related Art

Conventional wigs have been made in a standardized manner by transplanting hair into a cap-shaped transplant portion while maintaining the entire wig shape with the transplant portion. However, the wigs made in such manner can make hair rich but cannot help in forming the head shape.

In the case of users having a flat head shape where the back of the head is sunken, they have tried to overcome the head shape by volumizing the hair on the crowns of their heads at a beauty salon. However, this volumizing has a problem that the hair on the crown gets calmed down over time.

Korean Patent No. 0844139 (Patent No. Jun. 30, 2008) which was filed and registered by the present applicant is characterized in that, as illustrated in FIG. 1, a plurality of hair lines are formed on the back of the head and artificial hair or natural hair is transplanted into the hair lines, thereby forming natural hair, and the hair lines are lifted up by users' own hair, thereby improving the volume-up effect.

However, the above technique merely maintains the volume-up effect temporarily, but the volume-up effect gets lost over time due to the weight of the hair lines and the artificial hair or natural hair transplanted into the hair lines.

### CITATION LIST

### Patent Literature

Patent Literature 1: KR 10-0844139 B1 Jun. 30, 2008

### SUMMARY OF THE INVENTION

The present invention has been created to solve the problem of the conventional technology. The present invention relates to a hairwear equipped with hair lines having multi-layer structures, wherein by virtue of the structure where one or more multi-layered hair lines protruding onto the top of a reference plane made by a front portion and base hair lines or a cap-shaped transplant portion are formed, a space between the reference plane or the cap-shaped transplant portion and the multi-layered hair lines is secured, and the multi-layered hair lines are made of a material capable of fixing the shape and the position, thereby making it possible to fix the shape and position of the multi-layered hair lines and further improving the volume-up function according to the user's need.

In order to achieve the above object, the hairwear according to the present invention comprises a front portion consisting of a skin color portion, and a clip fixing portion formed at both sides of the skin color portion; and one or more base hair lines both ends of which are fixed and combined to the front portion, wherein the front portion and the base hair lines are formed to make a reference plane, characterized in that one or more multi-layered hair lines both ends of which are fixed and combined to the front portion and which protrude onto the top of the reference plane made by the front portion and the base hair lines are formed, and that the multi-layered hair lines are made of a material capable of fixing the shape and the position, so that a space between the reference plane made by the front portion and the base hair lines and the multi-layered hair lines protruding onto the top of the reference plane can be secured or the position of the multi-layered hair lines is fixed so as to easily make the hair volume-up.

Meanwhile, the hairwear according to the present invention is hairwear with a cap-shaped transplant portion into which artificial hair or natural hair can be transplanted, characterized in that one or more multi-layered hair lines both ends of which are fixed and combined to the transplant portion and which protrudes onto the top of the transplant portion are formed, and that the multi-layered hair lines are made of a material capable of fixing the shape and the position, so that a space between the cap-shaped transplant portion and the multi-layered hair lines protruding onto the top of the transplant portion can be secured or the position of the multi-layered hair lines is fixed so as to easily make the hair volume-up.

The multi-layered hair lines formed on the hairwear according to the present invention may comprise one or more partial hair lines for forming partial volume-up, characterized in that the partial hair lines can prevent the multi-layered hair lines, which are positioned on the top of the partial hair lines, from sagging.

### BRIEF DESCRIPTION OF THE DRAWINGS

The above and other objects, features and advantages of the present invention will be more apparent from the following detailed description taken in conjunction with the accompanying drawings, in which:

FIG. 1 shows the hairwear according to the conventional technology;

FIG. 2 shows the reference plane of the hairwear applied to the present invention;

FIGS. 3 and 4 show one or more multi-layered hair lines protruding onto the top of the reference plane made by the front portion and the base hair lines according to one embodiment of the present invention;

FIGS. 5 and 6 show one or more multi-layered hair lines protruding onto the top of the cap-shaped transplant portion according to another embodiment of the present invention; and

FIG. 7 is to explain the hairwear and the partial hair lines in which the multi-layered hair lines according to the present invention are actually implemented.

### DETAILED DESCRIPTION OF THE PREFERRED EMBODIMENT

Hereinafter, with reference to the drawings attached herewith, the preferred embodiments according to the present invention will be explained in more detail.

The term “hairwear” used in the present invention is used to basically represent the function of considering the forming of the head shape, unlike the conventional wig concepts in which the term has been used to supplement lost hair, etc. For instance, the term is to include the function of volumizing the hair on the crown of the head, which is the top of the back of the head, so as to make it possible to reshape the back of the head, and also to include the function of securing a space where a device used for gradually developing brain engineering, e.g. a small-size device for measuring users' brain waves to measure the abnormality of the brain function or studying mental illness, etc. As such, the term is used to include the concept of making it possible to measure the brain function, etc. in daily life while small-size electronic devices including the form such as a chip are not exposed into the outside, as well as forming the head shape.

Artificial hair or natural hair will be transplanted into the skin color portion, the base hair lines and the multi-layered hair lines out of the front portion of the present invention. However, in order to help in easily understanding the main characteristics of the present invention, i.e. the effects of securing the space and forming the head shape due to volume-up, the present invention will be explained with reference to the drawing showing the head where the artificial hair or natural hair is not transplanted.

Out of the front portion of the present invention, the clip fixing portion is to fix clips so that the hairwear can be fixed on the head of the user. As the clips are combined to the hair of the user while the clips are fixed inside of the clip fixing portion, the hairwear is fixed on the head of the user. Out of the front portion, the skin color portion and the clip fixing portion can be consecutively formed, and they can also be joined to each other by a connection portion 13 for improving the productivity, etc.

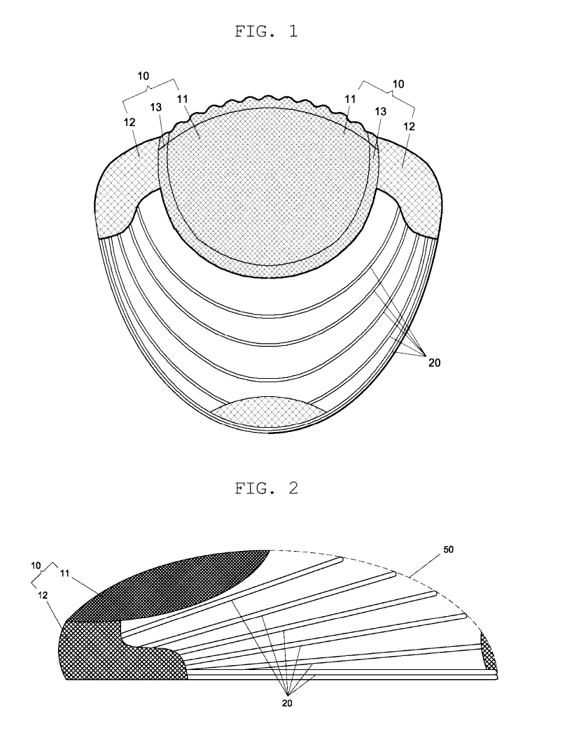

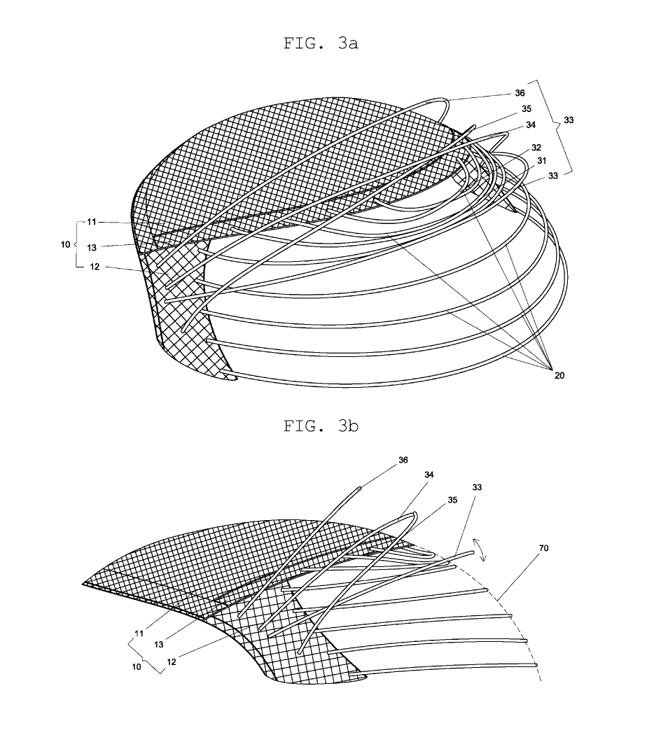

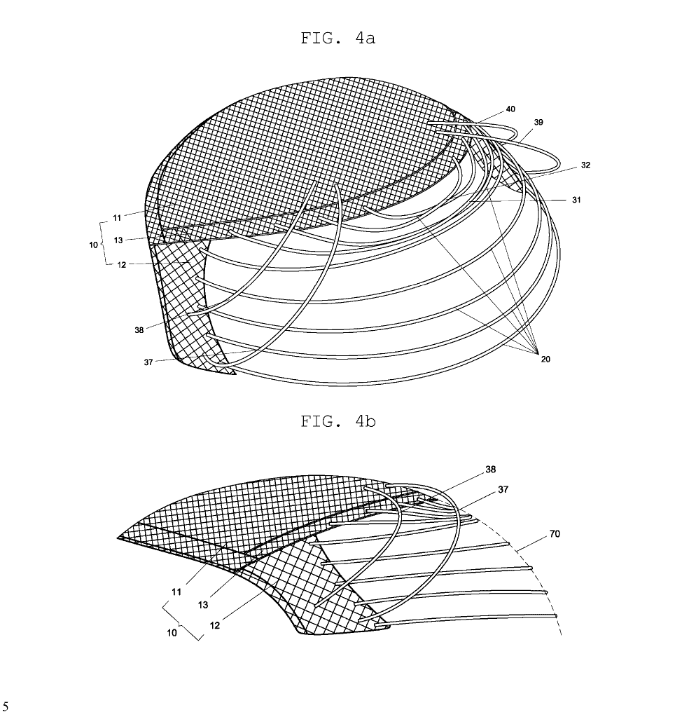

FIG. 2 shows the reference plane 70 of the hairwear 100 applied to the present invention. This figure shows the shape when viewing FIG. 1 from the side. To be specific, FIG. 2 illustrates that according to one embodiment of the present invention, the front portion 10 consisting of the skin color portion 11 and the clip fixing portion 12 formed at both sides of the skin color portion, and one or more base hair lines both ends of which are fixed and combined to the front portion make the reference plane 70.

According to the present invention, it can be understood that the hair lines positioned on the top of the reference plane, i.e. outside from the head with respect to the reference plane, are multi-layered hair lines, wherein the multi-layered hair lines are positioned on the upper part than the reference plane, thereby forming the user's head shape.

FIGS. 3 and 4 show one or more multi-layered hair lines 30 protruding onto the top from the reference plane 70 made by the front portion 10 and the base hair lines 20 according to one embodiment of the present invention. FIGS. 3a and 4a are the perspective views, and FIGS. 3b and 4b are the side views. The embodiments of FIGS. 3 and 4 illustrate that the multi-layered hair lines are formed as the 1st multi-layered hair line through the 6th multi-layered hair line, and the 1st multi-layered hair line, the 2nd multi-layered hair line and the 7th multi-layered hair line through the 10th multi-layered hair line, respectively. Herein, one or more multi-layered hair lines can be formed, but the number of the multi-layered hair lines is not particularly limited.

FIG. 3 shows the embodiment in which the ends of one sides of the 3rd multi-layered hair line through the 6th multi-layered hair line 33, 34, 35, 36 are fixed to any one side of the clip fixing portion 12 constituting the front portion, and the ends of the other sides are fixed to the other side of the clip fixing portion.

Referring to FIG. 3, including the 1st multi-layered hair line 31 and the 2nd multi-layered hair line 32, which are positioned on the upper part than the reference plane, the 3rd multi-layered hair line through the 6th multi-layered hair line 33, 34, 35, 36 all form the multi-layered hair lines. Referring to FIG. 3, as the 1st multi-layered hair line and the 2nd multi-layered hair line are formed adjacent to the reference plane, it can be used for the function of forming the head shape by volume-up, rather than the function of securing the space. In the case of the 3rd multi-layered hair line through the 6th multi-layered hair line, due to the characteristic of maintaining the state where the hair lines are positioned from the reference plane 70 at a constant distance, the function of securing the space can be achieved in addition to the function of forming the head shape by volume-up, so that it can be easily applied for the installation of small-size electronic devices for brain engineering. That is, if a small-size electronic device for brain engineering study is attached or installed on the space between the reference plane and the 3rd through 6th multi-layered hair lines, there are advantages that not only studies by the temporary measurement in the limited space such as medical facilities but also various study results occurring during the natural activities in daily life can be obtained without exposing the electronic device, etc., which are installed inside of the space of the hairwear into the outside.

FIG. 4 shows the embodiment in which the ends of one sides of the 7th multi-layered hair line through the 10th multi-layered hair line 37, 38, 39, 40 are fixed to any one side of the clip fixing portion 12 constituting the front portion, and the ends of the other sides are fixed to the skin color portion constituting the front portion. Even by the structure of FIG. 4, the function of securing the space can be achieved in addition to the function of forming the head shape by volume-up, so it can be easily applied for the installation of small-size electronic devices for brain engineering.

In order to equip a small-sized electronic device securely or make it easy to secure the space and/or form the head shape by volume-up, the multi-layered hair lines may be made of a material capable of maintaining the shape and fixing the position. The examples of the material for maintaining the shape and fixing the position include a wire consisting of metals such as iron or copper, but the materials are not limited thereto, and shape memory synthetic resins whose shape can be modified and which are capable of fixing the position and maintaining the shape as the user's modification, etc. also can be used as the material. However, in the case of using iron, it is preferable to use a stainless material which is rustproof even if it is exposed to the moisture of rain or snow.

The base hair lines are generally made of a fibrous material, and the multi-layered hair lines can also be made of a fibrous material.

Even if the multi-layered hair lines are made of a fibrous material, the both ends of the multi-layered hair lines are fixed on the appropriate position of the front portion, thereby making it possible to volume-up the hair and secure the space. However, in this case, when the users desire to change the position of the multi-layered hair lines as needed, there is a possibility that the change could not be made freely as the user desires.

If the multi-layered hair lines are made of a material capable of maintaining the shape and fixing the position, it is possible to dispose the multi-layered hair lines in a desired position even in the state where the both ends of the multi-layered hair lines are fixed to the front portion without swing. For example, referring to FIG. 3b, in case where the 3rd multi-layered hair line through the 6th multi-layered hair lines 33, 34, 35, 36 are made of a material capable of maintaining the shape and fixing the position, the securing can be possible in any position the user desires in the state where the 3rd multi-layered hair line 33 is freely moved in an arrow direction, thereby making it possible to form the head shape more easily and in various shapes.

In case where the 1st multi-layered hair line 31 and the 2nd multi-layered hair line 32, which are formed adjacent to the reference plane, are also made of a material capable of maintaining the shape and fixing the position, there are advantages that not only the head shape can be formed more easily, but also the space from the 3rd through 6th multi-layered hair lines 33, 34, 35, 36 or the 7th through 10th multi-layered hair lines 37, 38, 39, 40, which are formed at a distance, can be maintained more securely, so that small-size electronic devices, etc. can be installed stably.

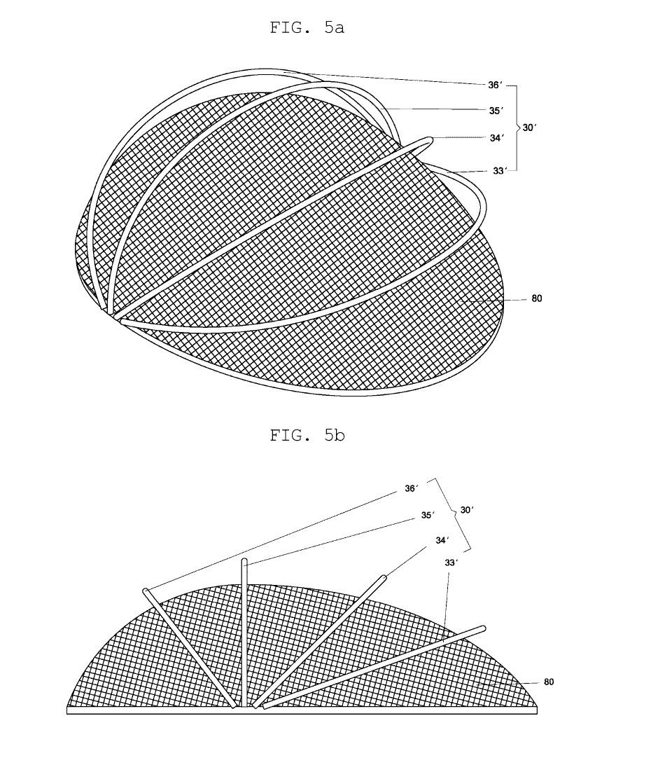

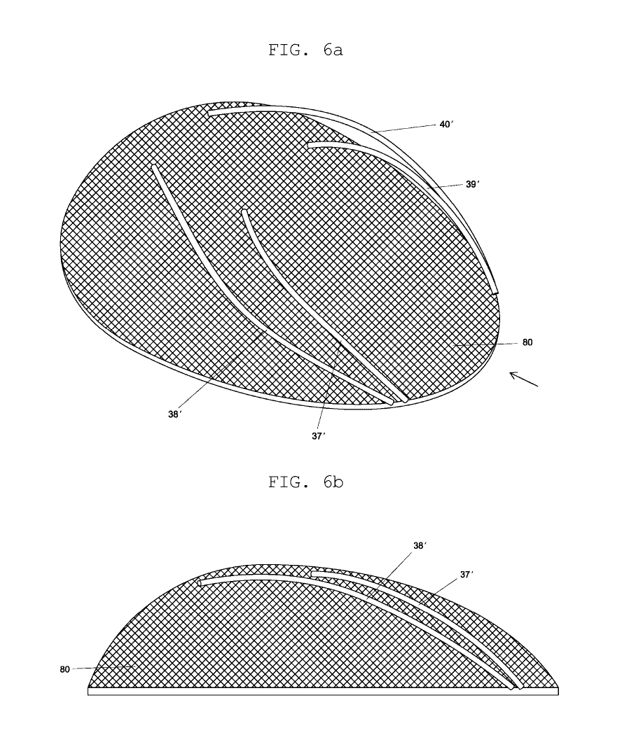

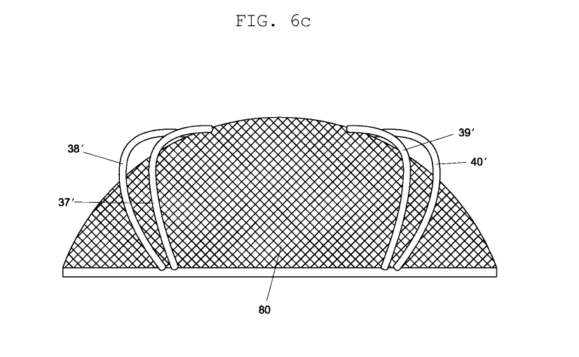

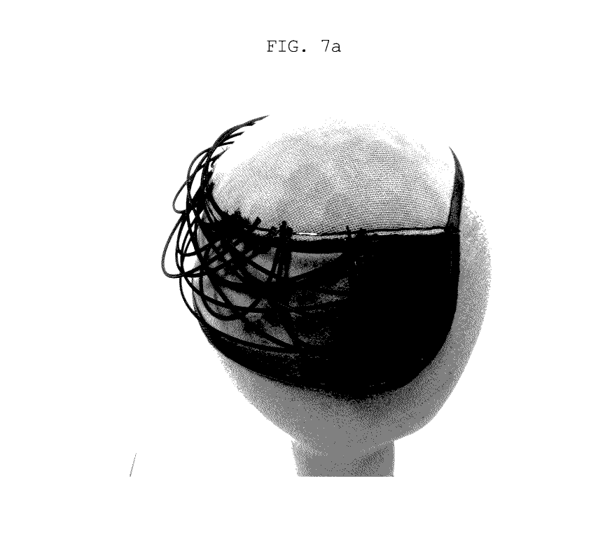

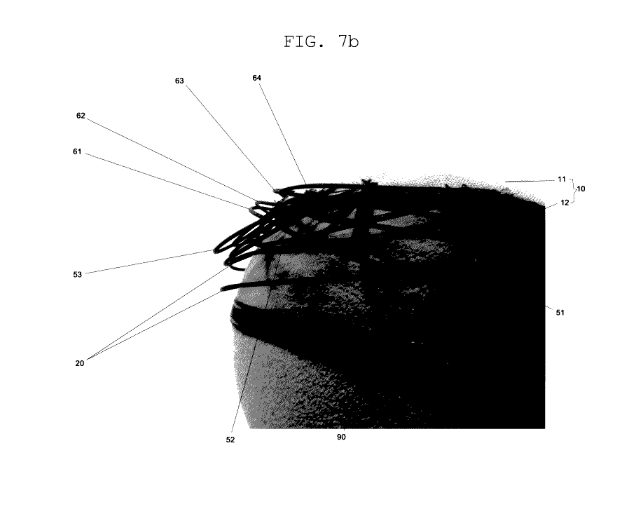

FIGS. 5 and 6 show one or more multi-layered hair lines 30′ protruding onto the top of the cap-shaped hair portion 80 according to another embodiment of the present invention. FIGS. 5a and 6a are the perspective views, and FIGS. 5b and 6b are the side views. In addition, FIG. 6c shows the shape when viewing the perspective views of FIG. 6a from the arrow direction.

The embodiments of FIGS. 5 and 6 differ from those of FIGS. 3 and 4 only in the constitution making the reference plane, but the functions of securing the space and forming the head shape by the volume-up can be achieved in the same manner. The embodiments of FIGS. 5 and 6 illustrate that the multi-layered hair lines are formed as the 11th multi-layered hair line through the 6th multi-layered hair line. Herein, one or more multi-layered hair lines can be formed, but the number of the multi-layered hair lines is not particularly limited.

FIG. 5 illustrates that the ends of one sides of the 13th multi-layered hair line through the 16th multi-layered hair line 33′, 34′, 35′, 36′ are fixed to any one side of the cap-shaped transplant portion 80, and the ends of the other sides are fixed to the other opposite side of the transplant portion. The position of the transplant portion on which the ends of the both sides of the 11th multi-layered hair line through the 14th multi-layered hair line may be a site positioned at the top of the both ears of the user, but the position is not particularly limited in the present invention, and the position may be a position across the front plane and the rear plane of the head of the user as needed. In the case of the 13th multi-layered hair line through the 16th multi-layered hair line, due to the characteristic of maintaining the state where the hair lines are positioned from the cap-shaped transplant portion 80 at a constant distance, the function of securing the space can be achieved in addition to the function of forming the head shape by volume-up, so that it can be easily applied for the installation of small-size electronic devices for brain engineering.

FIG. 6 shows the embodiment in which the ends of one sides of the 17th multi-layered hair line through the 20th multi-layered hair line 37′, 38′, 39′, 40′ are fixed to any one side of the cap-shaped transplant portion 80, and the ends of the other ends are fixed on the other side of the transplant portion, not the opposite side. Even by the structure of FIG. 6, the function of securing the space can be achieved in addition to the function of forming the head shape by volume-up, so it can be easily applied for the installation of small-size electronic devices for brain engineering.

The embodiments of FIGS. 5 and 6 illustrate that the multi-layered hair lines are formed as the 13th multi-layered hair line through the 16th multi-layered hair line, and the 17th multi-layered hair line through the 20th multi-layered hair line, respectively. Herein, one or more multi-layered hair lines can be formed, but the number of the multi-layered hair lines is not particularly limited.

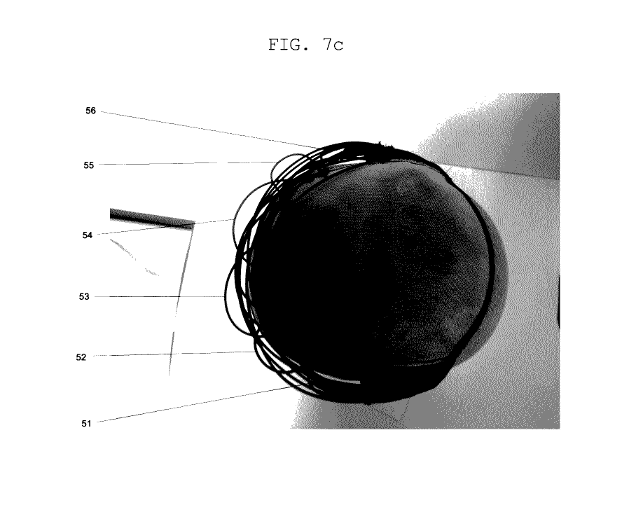

FIG. 7 is to explain the hairwear and the partial hair lines in which the multi-layered hair lines according to the present invention are actually implemented. FIG. 7a shows the shape that the hairwear according to the present invention is worn on the head of the model, and FIG. 7b shows the partially enlarged shape to explain the partial hair lines out of the multi-layered hair lines applied to the present invention.

Referring to FIG. 7b, it can be understood that the rear plane band 90, together with the front portion 10 consisting of the skin color portion 11 and the clip fixing portion 12, makes the reference plane, wherein partial hair lines 51, 52, 53 and upper hair lines 61, 62, 63, 64 are formed on the top of the reference plane. The partial hair lines are for partial volume-up as the user's need, and one or more partial hair lines, which are positioned on the top of the reference plane, are formed on a portion of the hairwear, and preferably, two or more partial hair lines are consecutively formed from one side of the hairwear to the other side, and the partial hair lines can also be put one upon another depending on the situation. FIG. 7(c) shows the shape where the plurality of partial hair lines 51 to 56 are formed, and shows the shape that any one partial hair line 52 and the other neighboring partial hair line 53 are superposed over each other. Referring to FIG. 7b, it can be understood that upper hair lines 61, 62, 63, 64 are formed on the top of the partial hair lines 51, 52, 53. If the multi-layered hair lines consist of the partial hair lines and the upper hair lines formed on the top of the partial hair lines, the upper hair lines can be prevented from sagging by the partial hair lines positioned on the lower part.

The multi-layered hair lines according to the present invention may be made of a material capable of maintaining the shape and fixing the position, and may be made of a fibrous material which is not easy to maintain the shape and fix the position like the base hair line, as needed. For example, if the upper hair lines are made of a fibrous material and the partial hair lines are made of a material capable of maintaining the shape and fixing the position, the upper hair lines can be more easily prevented from sagging by securing the shape and the position of the partial hair lines.

According to the present invention, due to the structure where one or more multi-layered hair lines protruding onto the top of the reference plane or the transplant portion are formed, the space between the reference plane or the transplant portion and the multi-layered hair lines is secured. In addition, the multi-layered hair lines are made of a material capable of maintaining the shape and fixing the position, thereby not only making it possible to partially volumizing the hair in the constant position of the head as the user's need by the partial hairline but also preventing the multi-layered hair lines, which are positioned on the top of the partial hair lines, from sagging by the partial hairline, while the substantive volume-up effect of the head shape can be maintained persistently.

According to the present invention, there are the following advantages: By virtue of the structure where one or more multi-layered hair lines protruding onto the top of the reference plane made by the front portion and the base hair lines or the cap-shaped transplant portion are formed, a space between the reference plane or the cap-shaped transplant portion and the multi-layered hair lines is secured, and it is possible to install small-size electronic devices related to brain engineering in the space, such as a Neuro biofeedback device for measuring user's brain waves or studying the moderns' mental illness or brain propensity. Further, since the multi-layered hair lines are made of a material capable of maintaining the shape and fixing the position, the substantive volume-up effect of the head shape can be maintained persistently. In addition, not only the partial hair lines make it possible to form partial volume-up in a constant position of the head as the user's need, but also the partial hair lines can prevent the multi-layered hair lines, which are positioned on the top of the partial hair lines, from sagging.

While the present invention has been described with respect to the specific embodiments, it will be apparent to those skilled in the art that various changes and modifications may be made without departing from the spirit and scope of the invention as defined in the following claims.

1. A hairwear comprising a front portion comprised of a skin color portion, and a clip fixing portion formed at both sides of the skin color portion; and one or more base hair lines both ends of which are fixed and combined to the front portion, wherein the front portion and the base hair lines are formed to make a reference plane, characterized in that one or more multi-layered hair lines both ends of which are fixed and combined to the front portion and which protrude onto the top of the reference plane made by the front portion and the base hair lines are formed.

2. The hairwear according to claim 1, characterized in that the multi-layered hair lines are made of a material capable of fixing the shape and the position, so that a space between the reference plane made by the front portion and the base hair lines and the multi-layered hair lines protruding onto the top of the reference plane can be secured or the position of the multi-layered hair lines is fixed so as to easily make the hair volume-up.

3. A hairwear with a cap-shaped transplant portion into which artificial hair or natural hair can be transplanted, characterized in that one or more multi-layered hair lines both ends of which are fixed and combined to the transplant portion and which protrudes onto the top of the transplant portion are formed.

4. The hairwear according to claim 3, characterized in that the multi-layered hair lines are made of a material capable of fixing the shape and the position, so that a space between the cap-shaped transplant portion and the multi-layered hair lines protruding onto the top of the transplant portion can be secured or the position of the multi-layered hair lines is fixed so as to easily make the hair volume-up.

5. The hairwear according to claim 1, characterized in that the multi-layered hair lines comprise one or more partial hair lines for forming partial volume-up, and the partial hair lines can prevent the multi-layered hair lines, which are positioned on the top of the partial hair lines, from sagging.

6. The hairwear according to claim 2, characterized in that the multi-layered hair lines comprise one or more partial hair lines for forming partial volume-up, and the partial hair lines can prevent the multi-layered hair lines, which are positioned on the top of the partial hair lines, from sagging.

7. The hairwear according to claim 3, characterized in that the multi-layered hair lines comprise one or more partial hair lines for forming partial volume-up, and the partial hair lines can prevent the multi-layered hair lines, which are positioned on the top of the partial hair lines, from sagging.

8. The hairwear according to claim 4, characterized in that the multi-layered hair lines comprise one or more partial hair lines for forming partial volume-up, and the partial hair lines can prevent the multi-layered hair lines, which are positioned on the top of the partial hair lines, from sagging.
